(19) United States
(12) Patent Application Publication
(10) Pub. No.: US 2016/0081974 A1
(43) Pub. Date: Mar. 24, 2016

(54) COMPOSITION FOR PREVENTING OR TREATING EDEMA CONTAINING FLAVONOID COMPOUND

(71) Applicant: Research & Business Foundation SUNGKYUNKWAN UNIVERSITY, Suwon-si (KR)

(72) Inventors: Suk Chan LEE, Suwon-si (KR); Kye Won PARK, Suwon-si (KR); Kangsan ROH, Seoul (KR); Nojoon SONG, Suwon-si (KR); Jaehyun PARK, Suwon-si (KR)

(73) Assignee: Research & Business Foundation SUNGKYUNKWAN UNIVERSITY, Suwon-si (KR)

(21) Appl. No.: 14/857,927

(22) Filed: Sep. 18, 2015

(30) Foreign Application Priority Data
Sep. 18, 2014 (KR) 10-2014-0124557
Sep. 18, 2014 (KR) 10-2014-0124558

(51) Int. Cl. A61K 31/353; A61K 31/343; A61K 31/12

(52) U.S. Cl. CPC A61K 31/353; A61K 31/12; A61K 31/343

(57) ABSTRACT

Provided is a method of treating edema using a flavonoid compound or a pharmaceutically acceptable salt thereof, and by the method, acquired edema triggered due to cancer treatments can be cured. A composition containing the flavonoid compound is a chemical approach to a conventional physical treatment method, and when the composition is administered to an edema-induced mouse, a considerable reduction in size of edema is observed.

### CROSS-REFERENCE TO RELATED APPLICATION

This application claims priority to and the benefit of Korean Patent Application No. 10-2014-0124557, filed on Sep. 18, 2014 and Application No. 10-2014-0124558, filed on Sep. 18, 2014, the disclosure of which is incorporated herein by reference in its entirety.

### BACKGROUND

1. Field of the Invention

The present invention relates to a composition for preventing or treating edema, which contains a flavonoid compound.

2. Discussion of Related Art

The lymphatic system consists of various tissues and organs including special networks of lymphoid tissues having many lymphocytes, and lymphatic ducts carrying lymph. While a thymus component of various lymphoid organs is composed of epithelioreticular tissues, the lymphoid tissue generally has a network of reticular fibers (connective tissue-forming cells) and reticular cells (fixed macrophages).

While a lymphatic vessel has a similar structure to a blood vessel, it has a thin wall and many valves, joins to a large lymph duct, and includes lymph capillaries connecting to lymph nodes present in various sites of the body. Lymph nodes are most densely found in the face, neck, armpits, thoracic cavity, intestines, inguinal region, elbows and knees. Generally, thin superficial lymphatic vessels extend along a vein, while deep lymphatic vessels extend along an artery. The lymphatic vessels serve to provide lymph throughout the body, and return proteins leaked from capillaries to the cardiovascular system. Also, the lymphatic vessels carry lipids from the stomach to blood. In a cancer patient, lymphocytes serve to monitor and prevent the invasion of foreign cells, bacteria and cancer cells. Certain lymphocytes (T cells) release various substances to directly or indirectly destroy their invaders. Other lymphocytes (B cells) differentiate into plasma cells secreting antibodies with respect to foreign substances to aid in removing them. Lymph nodes have mesh-shaped fibrous tissues and serve as filters of foreign substances carried by lymph. In addition, lymph nodes produce lymphocytes, some of which are transferred from lymph to other parts of the body in the immune system. The spleen, thymus and tonsils are lymphoid organs that produce B cells, T cells and lymphocytes, as well as antibodies, to establish an immunological defense line of the lymphatic system.

Lymphedema is a condition of subcutaneous retention of lymph due to obstruction of lymphatic vessels, and penetration of obstructive tissues by macrophages induces inhibition of lymph stream by proteolytic degradation of obstructive proteins over a considerable amount of time. Radiation therapy that needs to be repeatedly performed in cancer patients has serious side effects as lymphedema is prolonged by continuously increasing obstruction of lymphatic drainage in a radiation region. Therefore, a new treatment method having no side effects is needed.

Particularly, after cancer patients undergo surgical operations due to side effects from the removal of lymph nodes to prevent the spread of cancer during intensive procedures including surgery, chemotherapy and radiation therapy for anticancer treatments, within 5 years, acquired edema is frequently found in their arms and legs.

Although acquired edema is a serious side effect from cancer treatment, which is found in more than 45% of patients with breast cancer that is the No. 1 cancer in women, and more than 10% of all cancer patients, no effective treatment method has been found in modern medicine yet. Since edema reduces a patient's quality of life and will to rehabilitate, it is necessary to carry out research on approaches to and targets for acquired lymphedema to the extent that cancer patients are increasing in modern society. However, there is still substantially no therapeutic agent for acquired edema.

Meanwhile, lipedema similar to lymphedema is a symptom also occurring as a sequela of anticancer treatments such as radiation therapy. Lipedema is also a factor in reduction of quality of life of a patient, and therefore to remove such edema, a method of directly removing an inner tissue of edema by, for example, lipectomy, has been conventionally used. However, with the conventional method, it is difficult to effectively remove edema and a patient experiences acute pain during treatment, and therefore the necessity for a better treatment method is emerging.

### SUMMARY OF THE INVENTION

The present invention is directed to providing a method of treating edema, which includes administering a flavonoid compound or a pharmaceutically acceptable salt thereof to a subject.

However, the technical problems to be accomplished by the present invention are not limited to the above-described problems, and other problems not described herein will be fully understood by those of ordinary skill in the art from the following descriptions.

In one aspect, the present invention provides a method of treating edema, which includes administering a flavonoid compound or a pharmaceutically acceptable salt thereof to a subject.

In one example embodiment of the present invention, the flavonoid compound or pharmaceutically acceptable salt thereof may inhibit mRNA expression of PPAR-γ or aP2.

In another example embodiment of the present invention, the flavonoid compound may be selected from the group consisting of butein, sulfuretin and isoflavone.

In still another example embodiment of the present invention, the edema may be lymphedema or lipedema.

In another aspect, the present invention provides a use of a flavonoid compound to prevent or treat edema.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 illustrates changes in edema size in experimental groups to which butein is continuously administered and PBS (phosphate buffered saline) administered after a lymphedema-induction procedure, and an untreated control group (negative control);

FIG. 2A illustrates mRNA expression of PPAR-γ as a marker of lymphedema in a butein-administered group compared to control groups;

FIG. 2B illustrates mRNA expression of aP2 as a marker of lymphedema in a butein-administered group compared to control groups;

FIG. 3 illustrates a change in a lymphedema-induced leg after butein is administered as a therapeutic agent by subcutaneous injection compared to control groups;

FIG. 4 illustrates images provided to compare swelling and thicknesses of both (right and left) legs between a normal mouse (negative control) and a lipedema-induced mouse (lipedema);

FIG. 5 illustrates comparative results of genes expressions for synthesizing hyaluronic acid, HAS1, HAS2, HAS3, and a receptor protein of the hyaluronic acid CD44 as markers in lipedema to demonstrate lipedema induction in a lipedema-induced mouse model;

FIG. 6 illustrates changes in edema size in experimental groups to which butein is continuously administered and PBS (phosphate buffered saline) administered after a lipedema induction procedure, and an untreated control group (negative control);

FIG. 7A illustrates mRNA expression of PPAR-γ as a marker of lipedema in a butein-administered group compared to control groups;

FIG. 7B illustrates mRNA expressions of aP2 as a marker of lipedema in a butein-administered group compared to control groups;

FIG. 8 illustrates changes in edema size in experimental groups to which sulfuretin is continuously administered and PBS (phosphate buffered saline) administered after a lymphedema induction procedure, and an untreated control group (negative control);

FIG. 9A illustrates mRNA expression of PPAR-γ in a sulfuretin-administered lymphedema group compared to controls;

FIG. 9B illustrates mRNA expression of aP2 in a sulfuretin-administered lymphedema group compared to controls;

FIG. 10 illustrates changes in edema size in experimental groups to which isoflavone is continuously administered and PBS (phosphate buffered saline) administered after a lymphedema induction procedure, and an untreated control group (negative control);

FIG. 11A illustrates mRNA expression of PPAR-γ in an isoflavone-administered lymphedema group compared to controls;

FIG. 11B illustrates mRNA expression of aP2 in an isoflavone-administered lymphedema group compared to controls; and

FIG. 12 illustrates changes in edema size in experimental groups to which sulfuretin or isoflavone are administered compared to PBS administered group after lymphedema induction, and an untreated control group.

### DETAILED DESCRIPTION OF EXEMPLARY EMBODIMENTS

In an effort to develop a new method capable of treating edema, the inventors found that injection of a composition containing a flavonoid compound has an effect of treating edema, and thus completed the present invention.

Accordingly, the present invention provides a method of treating lymphedema using a flavonoid compound or a pharmaceutically acceptable salt thereof.

In the present invention, the flavonoid compound may be butein, sulfuretin or isoflavone.

The structure of butein is represented by the following Formula 1.

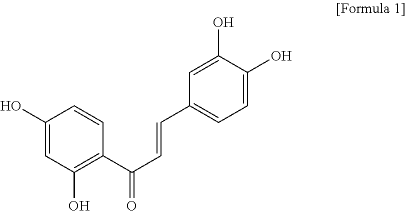 [SMILES: O=C(/C=C/c1ccc(O)c(O)c1)c1ccc(O)cc1O]

The structure of sulfuretin is represented by the following Formula 2.

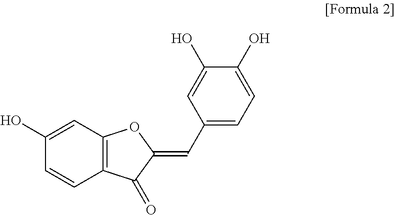 [SMILES: O=C1/C(=C/c2ccc(O)c(O)c2)Oc2cc(O)ccc21]

The structure of isoflavone is represented by the following Formula 3.

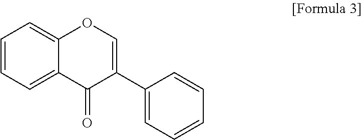 [SMILES: O=c1c(-c2ccccc2)coc2ccccc12]

In the present invention, the flavonoid compound may treat edema by reducing mRNA expression of PPAR-γ or aP2.

In the present invention, edema refers to a condition of excessive storage of fluids such as lymph in tissues. The type of edema may be lymphedema or lipedema, and is not limited as long as a disease shows a symptom of excessive storage of fluids in tissues.

The flavonoid compound of the present invention may be used in the form of a pharmaceutically acceptable salt, and as the salt, an acid addition salt formed by a pharmaceutically acceptable free acid is preferably used. The acid addition salt may be obtained from inorganic acids such as hydrochloric acid, nitric acid, phosphoric acid, sulfuric acid, hydrobromic acid, hydroiodic acid, azealic acid or phosphorous acid; and non-toxic organic acids such as aliphatic mono and dicarboxylates, phenyl-substituted alkanoates, hydroxy alkanoates and alkanedioates, aromatic acids, or aliphatic and aromatic sulfonic acids. Such pharmaceutically non-toxic salts include sulfate, pyrosulfate, bisulfate, sulfite, bisulfite, nitrate, phosphate, mono-hydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butene-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxy-butyrate, glycolate, malate, tartrate, methane sulfonate, propane sulfonate, naphthalene-l-sulfonate, naphthalene-2-sulfonate, or mandelate.

The acid addition salt according to the present invention may be prepared by a conventional method, for example, dissolving the flavonoid compound in an aqueous acid, and precipitating a water-miscible organic solvent using, for example, methanol, ethanol, acetone or acetonitrile.

The acid addition salt according to the present invention may be prepared by heating an equal amount of the flavonoid compound and an acid in water or alcohol, and then dehydrating the resultant mixture through evaporation or filtering the educed salt by means of suction.

In addition, the pharmaceutically acceptable metal salt may be prepared using a base. An alkali metal or alkali earth metal salt may be obtained by, for example, dissolving a compound in an excessive amount of an alkali metal hydroxide or alkali earth metal hydroxide solution, filtering an insoluble compound salt, and dehydrating the remaining solution through evaporation. Here, a sodium, potassium or calcium salt is pharmaceutically appropriate for the metal salt. Also, a silver salt corresponding to the metal salt is obtained by a reaction between an alkali metal or alkali earth metal salt and a suitable silver salt (e.g., silver nitrate).

Also, the flavonoid compound of the present invention includes all salts, hydrates and solvates that can be prepared by conventional methods, in addition to the pharmaceutically acceptable salts.

The addition salt according to the present invention may be prepared by a conventional method, for example, dissolving the flavonoid compound in a water-miscible organic solvent such as acetone, methanol, ethanol or acetonitrile, and adding an excessive organic acid or an inorganic acid solution to allow precipitation or crystallization. Subsequently, to obtain the addition salt, the solvent or excessive acid may be evaporated to dehydrate the mixture, or the educed salt may be filtered by means of suction.

In the present invention, the term “subject” refers to an object with a disease to be treated, and is more specifically, a mammal such as a human or a non-human primate, a mouse, a rat, a dog, a cat, a horse, or a cow. In addition, the term “pharmaceutically effective amount” used herein may vary depending on a patient's body weight, age, sex, health conditions, diet, duration of administration, administration method, excretion rate, and severity of a disease, which is apparent to those of ordinary skill in the art.

The flavonoid compound of the present invention may be administered in the form of a pharmaceutical composition, and a preferable dose may vary depending on a patient's conditions, body weight, severity of a disease, dosage form, administration route, and duration, and may be suitably determined by those of ordinary skill in the art. However, the composition is preferably administered at 0.001 to 100 mg, and more preferably, 0.01 to 30 mg per kg of body weight once or multiple times a day. The flavonoid compound of the present invention may be contained at 0.0001 to 99 wt % with respect to the total weight of the composition.

Also, the flavonoid compound of the present invention may be administered in the form of a food composition, and the composition may contain various fragrances that can be used in a food composition, natural carbohydrates, nutrients, vitamins, minerals (electrolytes), synthetic and natural flavoring agents, coloring agents, flavor enhancers (cheese, chocolate, etc.), pectic acid or salts thereof, alginic acid or salts thereof, organic acids, protective colloidal thickeners, pH modifiers, stabilizers, preservatives, glycerin, alcohol, or carbonating agents used in soft drinks. In addition to the above substances, the composition may further contain natural fruit juice, and fruit flesh for preparing fruit and vegetable drinks. These ingredients may be used independently or in combination. The contents of the ingredients have no limit, but may be about 0.001 to 20 parts by weight with respect to 100 parts by weight of the composition of the present invention.

Also, the present invention may provide a use of the composition containing a flavonoid compound to treat edema.

In the present invention, it was confirmed that the sizes of lymphedema are reduced (refer to Examples 2 and 4) and mRNA expressions of PPAR-γ and aP2, which are known as lymphedema-related adipocyte markers, are reduced (refer to Example 3) by administering butein to lymphedema-induced animal models.

Also, in the present invention, it was confirmed that the sizes of lipedema are reduced (refer to Example 6) and mRNA expressions of PPAR-γ and aP2, which are known as lipedema-related adipocyte markers, are reduced (see Example 7) by administering butein to lipedema-induced animal models.

Also, in the present invention, it was confirmed that the sizes of lymphedema are reduced (refer to Examples 8, 10 and 12) and mRNA expressions of PPAR-γ and aP2, which are known as lymphedema-related adipocyte markers, are reduced (refer to Examples 9 and 11) by administering sulfuretin and isoflavone as the flavonoid compounds to lymphedema-induced animal models.

Hereinafter, exemplary examples will be provided to help in understanding of the present invention. However, the following examples are merely provided to facilitate understanding of the present invention, and the scope of the present invention is not limited to the following examples.

### EXAMPLES

### Example 1

### Preparation of Lymphedema-Induced Animal Models

As mice, Outbred Mice ICR originating from Charles River Laboratories (Wilmington, Mass.) were used. 3 μl of 2% methylene blue as a staining dye for staining lymph nodes and lymphatic vessels was injected into the top of a right foot. After the injection, the right foot was massaged for about 3 minutes to allow a flow of the staining dye through lymphatic vessels to stain the lymphatic system. Zoletil50 (0.6 mg/kg) and Rompun (0.4 mg/kg), which are commercially provided by Bayer Korea, were administered as anesthetic drugs at the maximum content of 100 μl by intraperitoneal injection.

Specifically, a mouse was anesthetized with the anesthetic drugs (Zoletil50, Rompun), and its chest was shaved to facilitate a surgical operation and suturing, and observation. A small amount (3 μl) of a 2% methylene blue solution was injected into the top of the right foot, and then the injected site was massaged for 3 minutes to allow the staining dye to spread well through the lymphatic vessels. To minimize inflammation which may be caused when lymph nodes in a groin region were excised and lymphatic vessels in a thigh were blocked, a minimum area of the right groin was incised open. Afterward, a superficial inguinal node and popliteal node, and a deep inguinal node, which were stained with methylene blue, were sequentially removed, and the stained lymphatic vessels in the right thigh were blocked using a soldering iron. Since 70 to 80% of the total lymphatic vessels were present in a subcutaneous region, the subcutaneous region in a suturing site was seared with the soldering iron to completely block the lymphatic system under the suturing site, and the right thigh was finally closed by suturing. About 3 days later, an increase in edema was confirmed.

### Example 2

### Confirmation of Lymphedema Treatment Effect of Butein

Each of PBS, hyaluronidase and butein was administered into an edema site of the mouse prepared in Example 1 at 15 mg per kg a day, and the size of edema was measured each day.

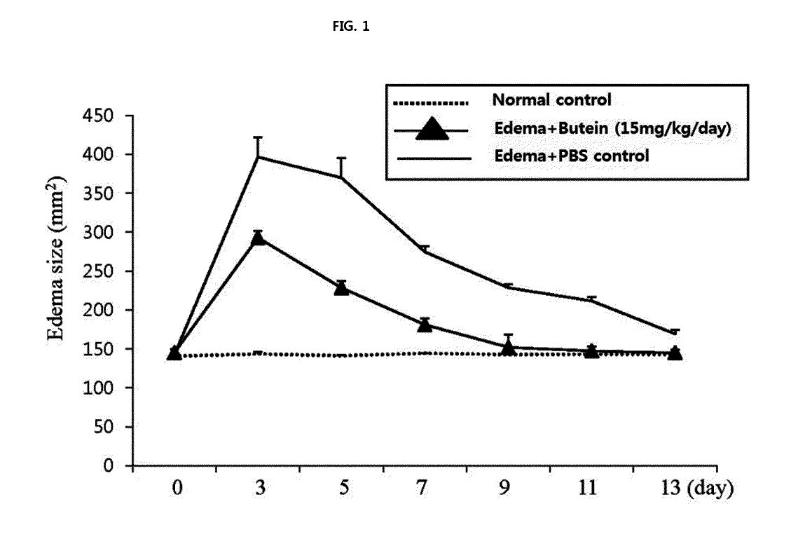

The measurement result is shown in FIG. 1. FIG. 1 shows the lymphedema inhibition of butein, and the size of the lymphedema-induced site, which is a thigh area present between the knee and the trunk, of the experimental animal was measured using a caliper. The cross-sectional area of lymphedema was calculated from the width and length of the thigh and recorded, and thus a change in size of lymphedema during observation was found.

It could be confirmed that, compared to a control group, when butein was treated, the size of lymphedema was significantly reduced (normal control: control group in which edema was not induced (n=3), edema+PBS: group into which PBS was injected after edema induction (n=4), and edema+butein: group into which butein was injected after edema induction (15 mg/kg/day) (n=5)).

### Example 3

### Confirmation of Effect of Butein on Decrease in Expression of Adipocyte Marker

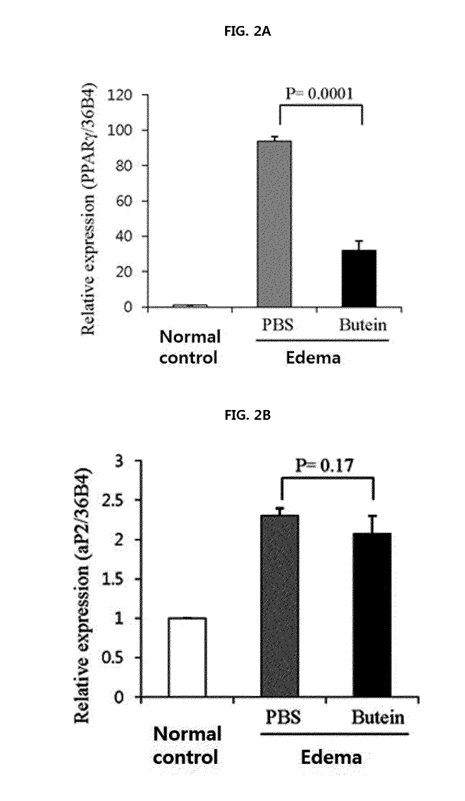

In this example, to investigate a mechanism for inducing reduction in lymphedema, expression of an adipocyte-related marker greatly found in edema was examined. Butein was administered into the animal in which edema was induced by lymph excision in Example 1 at a content of 15 mg per kg a day for 14 days. An edema tissue was collected, and mRNAs of adipocyte markers such as PPAR-γ and aP2 that are greatly found in edema were quantitatively analyzed by real time PCR, the result of which is shown in FIG. 2 (normal control: control group in which edema was not induced (n=3), edema+PBS: group into which PBS was injected after edema induction (n=4), edema+butein: group into which butein was injected after edema induction (15 mg/kg/day) (n=5)).

As the result of detecting mRNA expressions of PPAR-γ and aP2 as shown in FIG. 2, it could be confirmed that butein inhibits PPAR-γ and aP2 expression. Accordingly, it was confirmed that the inhibition of adipocytes by the butein treatment has an influence on edema reduction.

### Example 4

### Confirmation of Lymphedema Reduction Effect of Butein

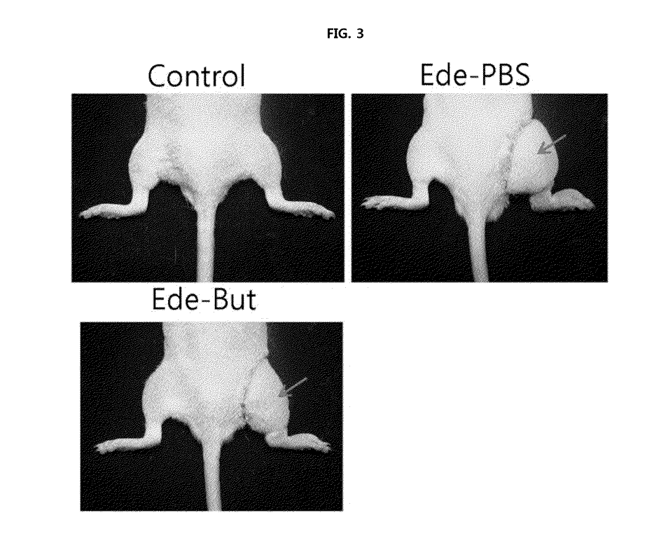

Butein (15 mg/kg/day) was administered into an animal in which lymphedema was induced by lymph excision in Example 1 by subcutaneous injection for 5 days, and the result is shown in FIG. 3. Referring to FIG. 3, it could be confirmed that, compared to a control group, when butein was treated, the size of lymphedema was significantly reduced. An edema-induced right leg is indicated by an arrow (control: edema-induced normal control (n=3), Ede-PBS: group into which PBS was injected after edema induction (n=4), Ede-But: group into which butein was injected after edema induction (15 mg/kg/day) (n=5)).

### Example 5

### Preparation of Lipedema-Induced Animal Models

5.1. Induction of Lipedema

Lymph nodes were removed from a mouse leg by a similar method to the method described in Example 1 to induce lipedema in the experimental animal, and an increase in edema was confirmed about 3 days after the removal of the lymph nodes.

5.2. Confirmation of Lipedema Induction

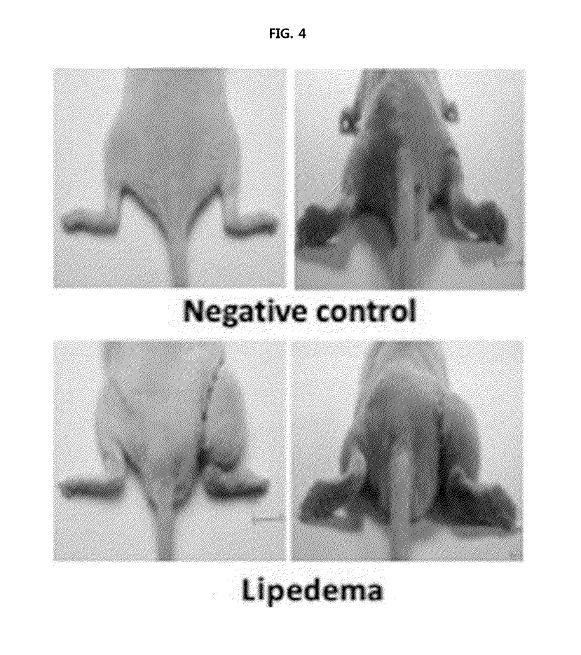

Referring to FIG. 4, comparing swelling and thicknesses of the right and left legs of a normal mouse (negative control, top) and a lipedema-induced mouse (lipedema, bottom), it can be seen that the leg of the lipedema-induced mouse was more swollen and became larger than that of the normal mouse.

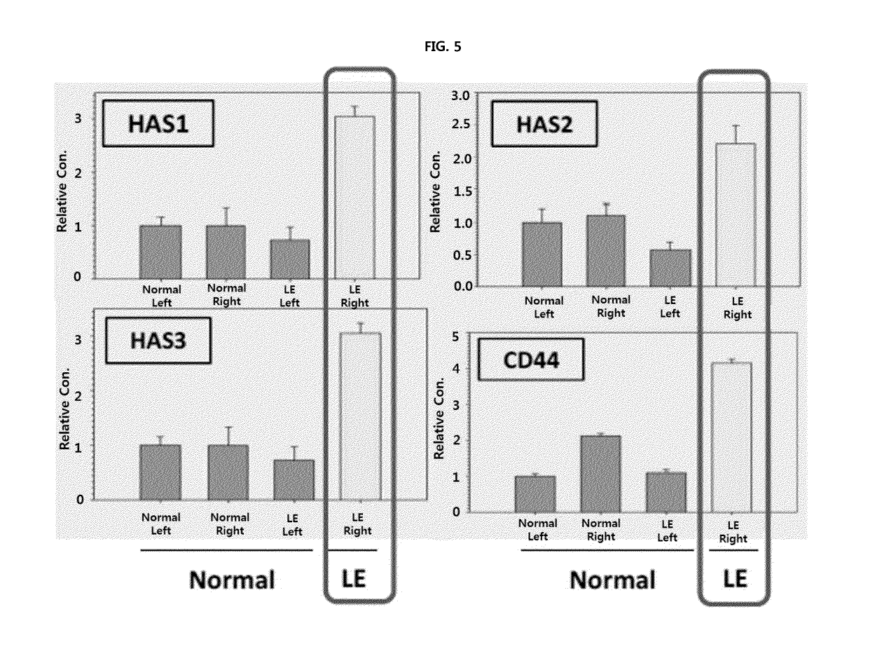

Also, to verify lipedema induction in the lipedema-induced mouse, expressions of genes for synthesizing hyaluronic acid accumulated by lipedema such as HAS1, 2 and 3, and a receptor protein of the hyaluronic acid, CD44, were compared, and the result is shown in FIG. 5.

Referring to FIG. 5, it can be confirmed that the gene expressions of HAS 1, 2 and 3 and CD44 were higher in the lipedema-induced tissue than in the normal tissue.

### Example 6

### Confirmation of Lipedema Treatment Effect of Butein

PBS (control) or butein (15 mg/kg/day) was injected into an edema site of the lipedema-induced mouse prepared in Example 5, and the edema size was measured every day.

A thigh area present between the knee and the trunk of the lipedema-induced site of the experimental animal was measured using a caliper. The cross-sectional area of lipedema was calculated from the width and length of the thigh and recorded, and thus a change in size of lipedema during observation was found.

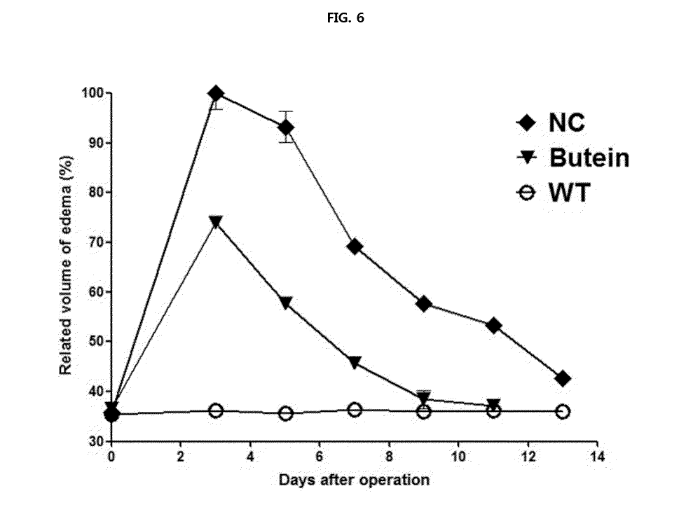

The measurement result is shown in FIG. 6. Referring to FIG. 6, it can be confirmed that the edema size in the butein-administered experimental group is more rapidly and greatly reduced than in the untreated experimental group.

### Example 7

### Confirmation of Effect of Butein on Decrease in Expression of Adipocyte Marker

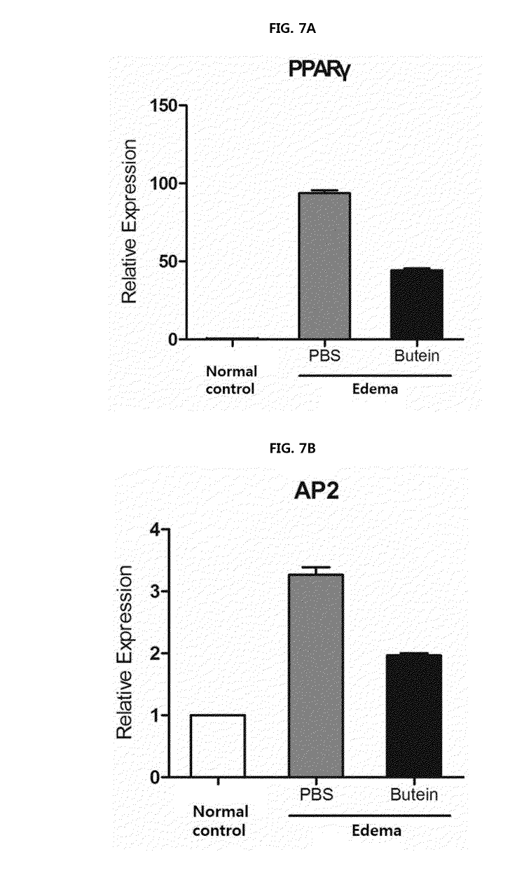

In this example, to investigate a mechanism for inducing the reduction of lipedema, expression of an adipocyte-related marker that is easily found in lipedema was examined. An edema tissue was isolated after butein was injected into lipedema for 14 days, and mRNAs of adipocyte markers, PPAR-γ and aP2, isolated from total RNA were quantified, and the result is shown in FIG. 7. Referring to FIG. 7, it could be confirmed that butein reduced the expression of PPAR-γ and aP2. Therefore, it can be seen that the inhibition of adipocytes by the butein administration has an influence on lipedema reduction.

### Example 8

### Confirmation of Lymphedema Treatment Effect of Sulfuretin

Each of PBS, hyaluronidase and sulfuretin (15 mg/kg/day) was injected into an edema site of the mouse prepared in Example 1, and the size of edema was measured every day.

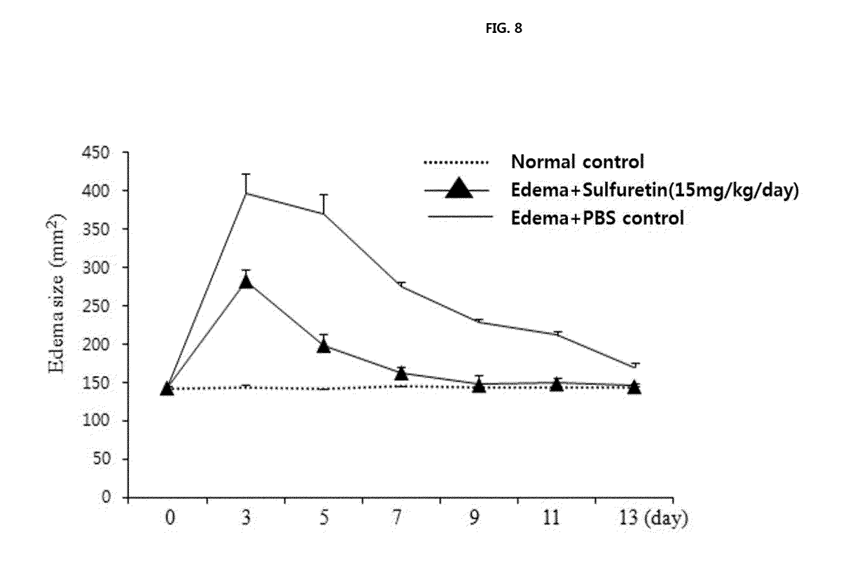

The measurement result is shown in FIG. 8. FIG. 8 illustrates the inhibition of lymphedema by sulfuretin, and the size measurement was performed on a thigh area present between the knee and the trunk of the lymphedema-induced site of the experimental animal using a caliper. The cross-sectional area of lymphedema was calculated from the width and length of the thigh and recorded, and thus a change in the lymphedema size that occurs in observation was found.

It could be confirmed that, compared to a control group, when sulfuretin was treated, the lymphedema size was significantly reduced (normal control: control group in which edema was not induced (n=3), edema+PBS: group into which PBS was injected after edema induction (n=4), edema+sulfuretin: group into which sulfuretin was injected after edema induction (15 mg/kg/day) (n=5)).

### Example 9

### Confirmation of Effect of Sulfuretin on Decrease in Expression of Adipocyte Marker

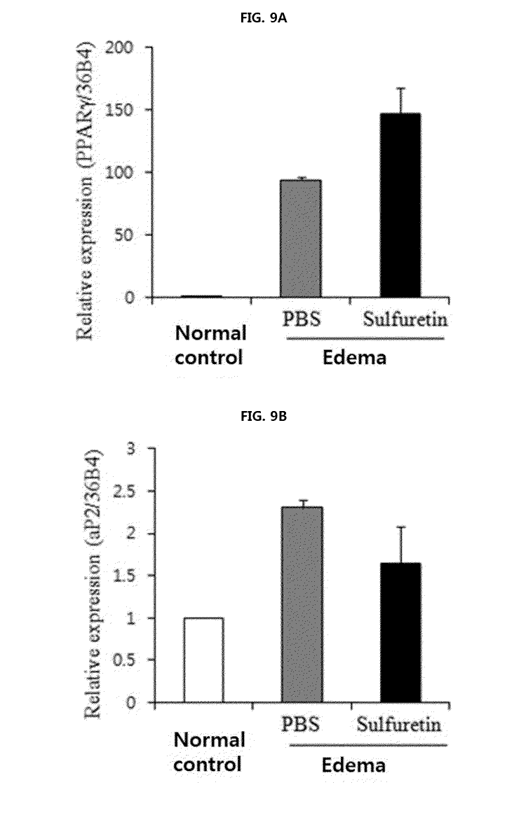

In this example, to investigate a mechanism for inducing reduction in lymphedema, expression of an adipocyte-related marker easily found in edema was examined. Sulfuretin was administered into the animal in which edema was induced by lymph excision in Example 1 at a content of 15 mg/kg a day for 14 days. An edema tissue was collected, and mRNAs of adipocyte markers such as PPAR-γ and aP2, which are greatly found in edema, were quantitatively analyzed by real time PCR, and the result is shown in FIG. 9 (normal control: control group in which edema was not induced (n=3), edema+PBS: group into which PBS was injected after edema induction (n=4), edema+sulfuretin: group into which sulfuretin was injected after edema induction (15 mg/kg/day) (n=5)).

Referring to FIG. 9, as the result of measuring relative concentrations of PPAR-γ and aP2, it was confirmed that sulfuretin inhibits aP2. Therefore, it was confirmed that the inhibition of adipocytes by the sulfuretin treatment has an influence on edema reduction.

### Example 10

### Confirmation of Lymphedema Treatment Effect of Isoflavone

Each of PBS, hyaluronidase and isoflavone (100 mg/kg/day) was injected into an edema site of the mouse prepared in Example 1, and the size of edema was measured every day.

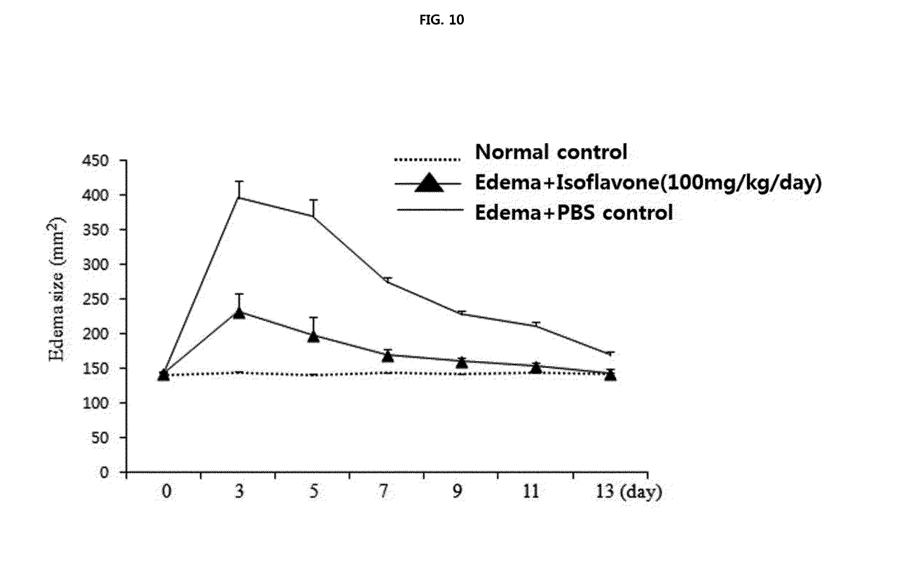

The measurement result is shown in FIG. 10. FIG. 10 illustrates the inhibition of lymphedema by isoflavone, and the size measurement was performed on a thigh area present between the knee and the trunk of the lymphedema-induced site of the experimental animal using a caliper. The cross-sectional area of lymphedema was calculated from the width and length of the thigh and recorded, and thus a change in the lymphedema size during observation was found.

It could be confirmed that, compared to a control group, when isoflavone was treated, the lymphedema size was significantly reduced (normal control: control group in which edema was not induced (n=3), edema+PBS: group into which PBS was injected after edema induction (n=4), edema+isoflavone: group into which isoflavone was injected after edema induction (100 mg/kg/day) (n=5)).

### Example 11

### Confirmation of Effect of Isoflavone on Decrease in Expression of Adipocyte Marker

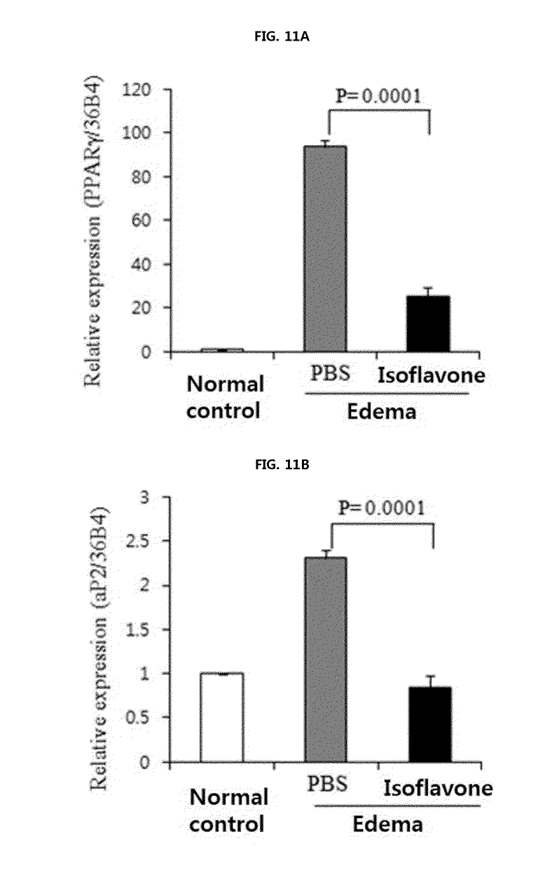

In this example, to investigate a mechanism for inducing reduction in lymphedema, expression of an adipocyte-related marker greatly found in edema was examined. Isoflavone was administered into the animal in which edema was induced by lymph excision in Example 1 at a content of 100 mg per kg a day for 14 days. An edema tissue was collected, and mRNAs of adipocyte markers such as PPAR-γ and aP2, which are greatly found in edema, were quantitatively analyzed by real time PCR, and the result is shown in FIG. 11 (normal control: control group in which edema was not induced (n=3), edema+PBS: group into which PBS was injected after edema induction (n=4), edema+isoflavone: group into which isoflavone was injected after edema induction (100 mg/kg/day)(n=5)).

Referring to FIG. 11, as the result of quantifying the mRNAs of PPAR-γ and aP2, it was confirmed that isoflavone inhibits PPAR-γ and aP2. Therefore, it was confirmed that the inhibition of adipocytes by the isoflavone treatment has an influence on edema reduction.

### Example 12

### Confirmation of Lymphedema Reduction Effects of Sulfuretin and Isoflavone

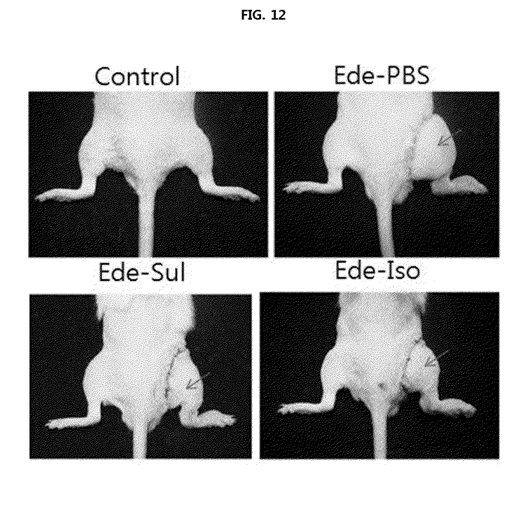

Each of sulfuretin (15 mg/kg/day) and isoflavone (100 mg/kg/day) was administered into an animal in which lymphedema was induced by lymph excision in Example 1 by subcutaneous injection for 5 days, and the result is shown in FIG. 12. Referring to FIG. 12, it was confirmed that, compared to a control group, when sulfuretin or isoflavone was treated, the size of lymphedema was significantly reduced. An edema-induced right leg in indicated by an arrow (control: edema-induced normal control (n=3), Ede-PBS: group into which PBS was injected after edema induction (n=4), Ede-Iso: group into which isoflavone was injected after edema induction (100 mg/kg/day)(n=5), Ede-Sul: group into which sulfuretin was injected after edema induction (15 mg/kg/day) (n=5)).

### Preparation Example 1

### Preparation of Pharmaceutical Composition

1. Preparation of Powder

Complex extract 20 ml

Lactose 100 mg

Talc 10 mg

A powder was prepared by mixing the above ingredients and filling an air-tight bag with the resultant mixture.

2. Preparation of Tablet

Complex extract 10 ml

Corn starch 100 mg

Lactose 100 mg

Magnesium stearate 2 mg

A tablet was prepared by mixing the above ingredients and pressing the resultant mixture according to a conventional method of preparing a tablet.

3. Preparation of Capsule

Complex extract 10 ml

Crystalline cellulose 3 mg

Lactose 14.8 mg

Magnesium stearate 0.2 mg

A capsule was prepared by mixing the above ingredients and filling a gelatin capsule with the resultant mixture according to a conventional method of preparing a capsule.

4. Preparation of Injection

Complex extract 10 ml

Mannitol 180 mg

Injectable sterilized distilled water 2974 mg

Na₂HPO₄2H₂O 26 mg

An injection was prepared by mixing the above ingredients at the above contents per ampoule (2 ml) according to a conventional method of preparing an injection.

5. Preparation of Liquid

Complex extract 20 ml

Isomerose 10 g

Mannitol 5 g

Distilled water suitable amount

A liquid was prepared by dissolving each of the above ingredients in distilled water, adding a suitable amount of lemon flavoring, mixing the resultant solutions, adding distilled water to the resultant product to have a final content of 100 ml, and filling a brown bottle with the resultant solution according to a conventional method of preparing a liquid.

### Preparation Example 2

### Preparation of Food

1. Preparation of Health Food

Complex extract 100 ml

Vitamin mixture suitable amount

Vitamin A acetate 70 g

Vitamin E 1.0 mg

Vitamin B1 0.13 mg

Vitamin B2 0.15 mg

Vitamin B6 0.5 mg

Vitamin B12 0.2 g

Vitamin C 10 mg

Biotin 10 g

Nicotinamide 1.7 mg

Folic acid 50 g

Calcium pantothenate 0.5 mg

Mineral mixture suitable amount

Ferrous sulfate 1.75 mg

Zinc oxide 0.82 mg

Magnesium carbonate 25.3 mg

Monopotassium phosphate 15 mg

Dipotassium phosphate 55 mg

Potassium citrate 90 mg

Calcium carbonate 100 mg

Magnesium chloride 24.8 mg

The vitamin and mineral mixtures were prepared by mixing relatively suitable ingredients for a health food as exemplary examples. but a mixing ratio of the ingredients may be arbitrarily changed. Granules were prepared by mixing the above ingredients according to a conventional method of preparing a health food, and the granules may be used to prepare a health food composition according to a conventional method.

2. Preparation of Health Drink

Complex extract 100 ml

Vitamin C 15 g

Vitamin E (powder) 100 g

Ferrous lactate 19.75 g

Zinc oxide 3.5 g

Nicotinamide 3.5 g

Vitamin A 0.2 g

Vitamin B1 0.25 g

Vitamin B2 0.3 g

Water suitable amount

The above ingredients were mixed according to a conventional method of preparing a health drink, heated with stirring for about 1 hour at 85° C., filtered and collected in a sterilized container. The resultant product was sealed and sterilized, and stored in a refrigerator until used to prepare the health drink composition of the present invention.

The composition was prepared by mixing relatively suitable ingredients for a high-palatability drink as an exemplary example, but a mixing ratio may be arbitrarily changed depending on regional or ethnic preferences such as a consumer group, a consuming country or a use.

In developed countries, the trend toward aging society, and guarantee of a healthy life and reduction in medical costs caused thereby are becoming important issues to both individuals and society, and products made of natural substances can be developed and distributed in the market as high value health functional foods and medicines in industrial aspects.

Cancer patients have been treated by methods such as surgical operations, chemotherapy, and radiation therapy, however, they are suffering from edema caused by such treatments, which becomes another difficulty after the cancer treatment. Edema is found in about 40% of the cancer patients, and particularly, found in patients with breast cancer with a high frequency after a surgical operation. However, there is still no treatment method for preventing, relieving and treating edema. While treatments frequently executed for patients with lymphedema and lipedema include Manual Lymph Drainage, compression bandaging, liposuction, etc., these treatments need to be applied repeatedly and are only temporary methods, not radical solutions to remove the cause. Therefore, lymphedema and lipedema recur. Also, due to the expected increase in cancer patients caused by aging society and the occurrence of edema caused thereby, economical and social costs to improve a patient's quality of life also increase.

As a consequence of injection of a flavonoid compound to each of lymphedema- and lipedema-induced animals, the volume and size of edema were shown to be significantly lower than those of a control group.

Accordingly, this showed that butein enables a reduction in expression of an adipocyte marker, which is found in edema, and thus can be used as an effective substance to prevent and treat lymphedema and lipedema.

Since the use of the composition of the present invention is a method of injecting a drug, unlike the conventional physical treatment method, it can lead to edema reduction without great pain, and is simple.

Moreover, since the method of the present invention does not badly impair other tissues in edema, considering a principle of the treatment method, there are few concerns of complications.

It would be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments and experimental examples described above are exemplary in all aspects, and are not limitative.

What is claimed is:

1. A method of treating edema, comprising:
administering a flavonoid compound or a pharmaceutically acceptable salt thereof to a subject.

2. The method of claim 1, wherein the flavonoid compound or the pharmaceutically acceptable salt thereof inhibits mRNA expression of PPAR-γ or aP2.

3. The method of claim 1, wherein the flavonoid compound is selected from the group consisting of butein, sulfuretin and isoflavone.

4. The method of claim 1, wherein the edema is lymphedema or lipedema.
